(19) United States
(12) Patent Application Publication
(10) Pub. No.: US 2025/0144262 A1
(43) Pub. Date: May 8, 2025

(54) AROMA DIFFUSER

(71) Applicant: SHENZHEN JINGXINTAI HOUSEWARE CO., LTD., Shenzhen (CN)

(72) Inventors: Jinshan ZOU, Shenzhen (CN); Shouyong ZHENG, Shenzhen (CN); Lingzhi LUO, Shenzhen (CN)

(21) Appl. No.: 18/541,994

(22) Filed: Dec. 15, 2023

(30) Foreign Application Priority Data
Nov. 4, 2023 (CN) 2023229878998

(51) Int. Cl. A61L 9/14; A61L 9/12

(52) U.S. Cl. CPC A61L 9/14; A61L 9/122; A61L 2209/134

(57) ABSTRACT

An atomization mechanism of an aroma diffuser includes an atomization shell and an essential oil bottle. The essential oil bottle is provided with a first cavity, and the atomization shell is provided with a second cavity. When the atomization mechanism is placed normally, pressure exerted by a ball on a spring can compress the spring, making a second communication hole of a leakage-proof cover open. When the atomization mechanism is tilted, pressure exerted by the ball on the spring is not sufficient to compress the spring, and the spring stretches to make the ball abut against at least a portion of the leakage-proof cover, so that the second communication hole of the leakage-proof cover is closed.

### TECHNICAL FIELD

The present invention relates to the technical field of environmental electrical appliances, in particular to an aroma diffuser.

### BACKGROUND TECHNOLOGY

Aromatherapy not only adds fragrance to the environment but also has health benefits such as calming the mind, combating fatigue, and inducing a sense of joy. An aroma diffuser can atomize liquid essential oils into mist, allowing aromatic mist to float in the air and gradually disperse into the environment, thereby filling the entire space. With improving living standards, aroma diffusers are increasingly being installed in homes, offices, and shops.

Aroma diffusers used in home environments are small and have low power, covering only a limited area. Therefore, it is difficult to apply to large public areas such as hotel lobbies, bank lobbies, office halls, supermarkets, airports, and train stations.

However, simply increasing the size and power of aroma diffusers will lead to a series of problems: 1. increasing the size and power of air pumps will result in excessive noise during operation; 2. increasing the size and power of air pumps will lead to a sharp increase in the consumption of essential oils; and when aroma diffusers tilt, a large amount of essential oils may leak out.

In summary, providing an adaptive structural solution for the enlargement of the aroma diffuser is an urgent problem that needs to be addressed.

### SUMMARY OF THE INVENTION

The purpose of the present invention is to provide an aroma diffuser that can eliminate a series of problems such as excessive noise, increased consumption of essential oils, and significant leaks of essential oils when the aroma diffuser is enlarged.

In order to achieve above purposes, the present invention provides following technical solutions: an aroma diffuser comprises an atomization mechanism, wherein the atomization mechanism comprises an atomization shell and an essential oil bottle, the essential oil bottle is provided with a first cavity, and the atomization shell is provided with a second cavity; the essential oil bottle and the atomization shell are connected in sequence from bottom to top, so that the first cavity and the second cavity communicate with each other; the atomization mechanism further comprises a leakage-proof cover, an elastic body and a ball; at the atomization shell is formed a leakage-proof accommodation cavity, at the leakage-proof accommodation cavity is provided a first communication hole communicated to the first cavity, at the leakage-proof cover is provided a second communication hole, and the anti-leakage cover shell is closed at an upper opening portion of the leakage-proof accommodation cavity so as to communicate the second communication hole to the second cavity; the elastic body and the ball are arranged inside the leakage-proof accommodation cavity and the leakage-proof cover from bottom to top, and an end of the elastic body abuts against an inner wall of the leakage-proof accommodation cavity, and another end of the elastic body abuts against the ball; and when the atomization mechanism is placed normally, pressure exerted by the ball on a spring compresses the spring to make the second communication hole of the leakage-proof cover open; when the atomization mechanism is tilted, pressure exerted by the ball on the spring is not sufficient to compress the spring, and the spring stretches to make the ball abut against at least a portion of the leakage-proof cover, so that the second communication hole of the leakage-proof cover is closed.

In above technical solutions, an opening portion of the leakage-proof cover forms a abutting portion matching with the ball; when the atomization mechanism is tilted, pressure exerted by the ball on the spring is not sufficient to compress the spring, and the spring extends to make the ball to abut against the abutting portion of the leakage-proof cover, thereby closing the second communication hole of the leakage-proof cover.

In above technical solutions, on a bottom inner wall of the leakage-proof accommodation cavity forms a support column for supporting the elastic body.

In above technical solutions, the atomization mechanism further comprises a nozzle assembly, the nozzle assembly comprises an atomization nozzle and an air intake nozzle; the atomization nozzle comprises a longitudinally arranged oil suction channel and a longitudinally arranged atomization channel arranged parallel to each other, and a horizontally arranged oil suction channel comprising two ends connected to the longitudinally arranged oil suction channel and the longitudinally arranged atomization channel respectively, and an bottom end portion of the longitudinally arranged atomization channel is provided with an atomization jet orifice; inside the air intake nozzle forms an air injection channel, the air intake nozzle penetrates into the longitudinally arranged atomization channel of the atomization nozzle, so that the air injection channel of the air intake nozzle and the atomization jet orifice of the longitudinally arranged atomization channel are aligned with each other, and between the air intake nozzle and the longitudinally arranged atomization channel of the atomization nozzle is a gap which serves as an atomization cavity; an airflow channel is formed in the atomization shell, an inlet of the airflow channel is located outside the atomization shell, and an outlet of the airflow channel faces the essential oil bottle; a free end of the air inlet nozzle is inserted into an outlet of the air flow channel of the atomization shell, so that the nozzle assembly is supported in the first cavity of the essential oil bottle, an oil suction pipe is inserted at and connected to the longitudinally arranged oil suction channel of the atomization nozzle, and the free end of the oil suction pipe enters the essential oil bottle.

In above technical solutions, an outlet end of the air injection channel is narrowed into a truncated cone shape or a cone shape.

In above technical solutions, the aroma diffuser further comprises an outer shell mechanism, an inner support frame mechanism and an air pump, wherein the inner support frame mechanism comprises a lower support frame and an upper support frame, and the lower support frame and the upper support frame are fixed to each other and accommodated in the outer shell mechanism; the air pump is fixed in the lower support frame, and an air outlet end of the air pump is connected into a silencing cavity, and a first joint is provided at the silencing cavity; the upper support frame is provided with a second joint, and the second joint is connected to the first joint of the silencing cavity through an air pipe; and the atomization shell of the atomization mechanism is adapted to be plugged into the upper support frame of the inner support frame mechanism, and the atomization shell of the atomization mechanism is plugged into and connected to the upper support frame of the inner support frame mechanism, so that, the free end of the second joint is connected to the inlet of the airflow channel of the atomization shell.

In above technical solutions, the silencing cavity is formed at a bottom portion of the upper support frame, an opening portion of the silencing cavity is covered with a silencing cavity sealing plug, and the air outlet end of the air pump is connected into the silencing cavity sealing plug and thereby into the silencing cavity.

In above technical solutions, between the air outlet end of the air pump and the silencing cavity sealing plug is provided an air outlet buffer body.

In above technical solutions, between the air pump and an inner wall of the lower support frame is provided an air pump buffer body.

In above technical solutions, the aroma diffuser further comprises batteries and a circuit board, and both the batteries and the circuit board are accommodated in the outer shell mechanism, and the batteries and the air pump are both electrically connected to the circuit board.

Compared with the prior art, the present invention has following beneficial effects: in the aroma diffuser of the present invention, through the coordination of the elastic body and the ball, when tilted, essential oils cannot pass through the leakage-proof cover, thereby preventing leaks from the diffuser, enhancing its safety during use. Moreover, by incorporating the silencing cavity, the air outlet buffer, and the air pump buffer, noise and vibration during air pump operation are eliminated, making the aroma diffuser quieter when running. Additionally, the gap serving as an atomization cavity between the air intake nozzle and the longitudinally arranged atomization channel of the atomization nozzle reduces the volume of the atomization cavity, making the diffuser more compact. Since the atomization jet orifice of the atomization nozzle is oriented towards inner portions of the essential oil bottle, which on one hand, effectively reduces noise and preventing wastage of essential oils, and on the other hand provides the advantage of lower aroma consumption. In conclusion, the present invention resolves a series of problems arising from enlargement and provides an adaptive structure for the diffuser's enlargement.

### BRIEF DESCRIPTION OF THE DRAWINGS

FIG. 1 is a perspective view of an aroma diffuser of the present invention;

FIG. 2 is a cross-sectional view of the aroma diffuser of the present invention;

FIG. 3 is an enlarged view of a part “A” in FIG. 2;

FIG. 4 is an enlarged view of a part “B” in FIG. 3; and

FIG. 5 is an exploded view of an atomization mechanism of the present invention.

The markups in the drawings are indicated as follows:
1—outer shell mechanism;
11—outshell body;
12—base;
2—inner support frame mechanism;
21—lower support frame;
211—air pump buffer body;
22—upper support frame;
221—silencing cavity;
2211—first joint;
222—silencing cavity sealing plug;
223—air outlet buffer body;
224—second joint;
3—atomization mechanism;
31—atomization shell;
311—air flow channel;
312—leakage-proof accommodation cavity;
313—first communication hole;
314—support column;
32—leakage-proof cover;
321—second communication hole;
322—abutting portion;
33—elastic body;
34—ball;
35—essential oil bottle;
36—atomization nozzle;
361—longitudinally arranged oil suction channel;
362—horizontally arranged oil suction channel;
363—longitudinally arranged atomization channel;
364—atomization jet orifice;
37—air intake nozzle;
371—first mounting portion;
372—rib portion;
373—second mounting portion;
374—air injection channel;
38—oil suction pipe;
4—air pump;
5—battery;
6—circuit board;
7—air pipe;
10—first cavity; and
20—second cavity.

### Specific Embodiments

The technical solutions in embodiments of the present invention will be clearly and completely describe portioned below with reference to the accompanying drawings in embodiments of the present invention. Obviously, described embodiments are only some embodiments of the present invention, rather than all of the embodiments. Based on the embodiments of the present invention, all other embodiments obtained by those of ordinary skill in the art without creative efforts shall fall within protection scope of the present invention.

An embodiment of the present invention provides an aroma diffuser for atomizing aromatherapy essential oils into mist and diffusing the same into the air.

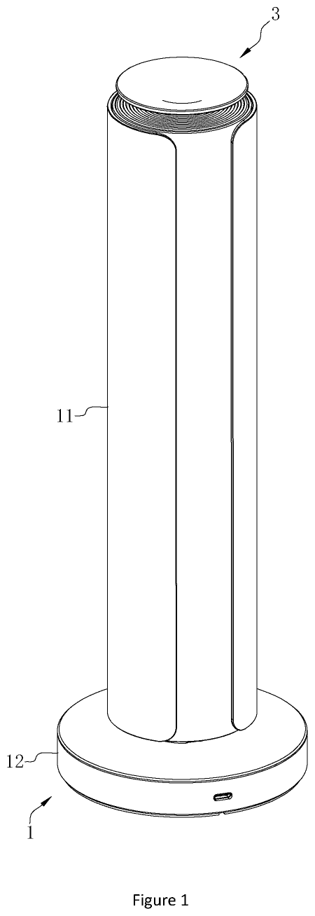

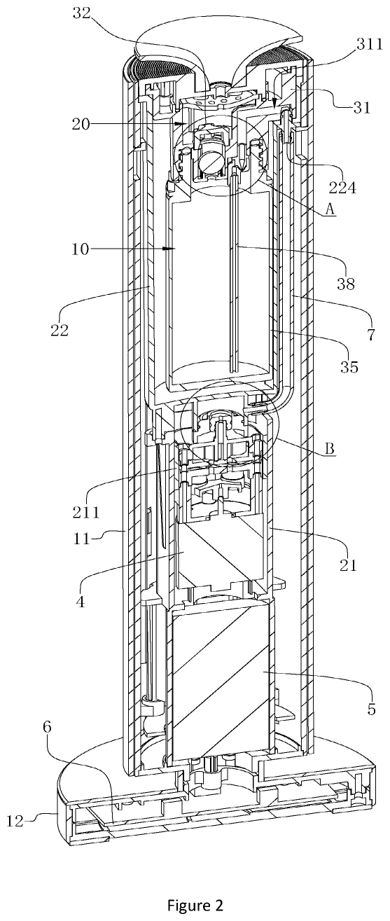

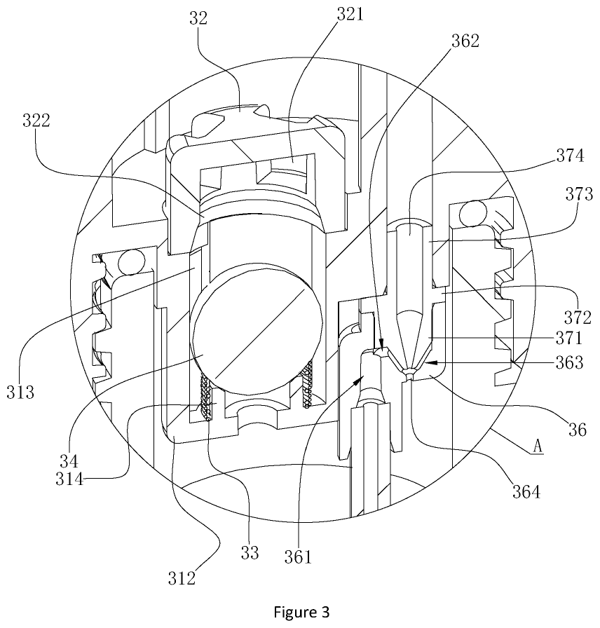

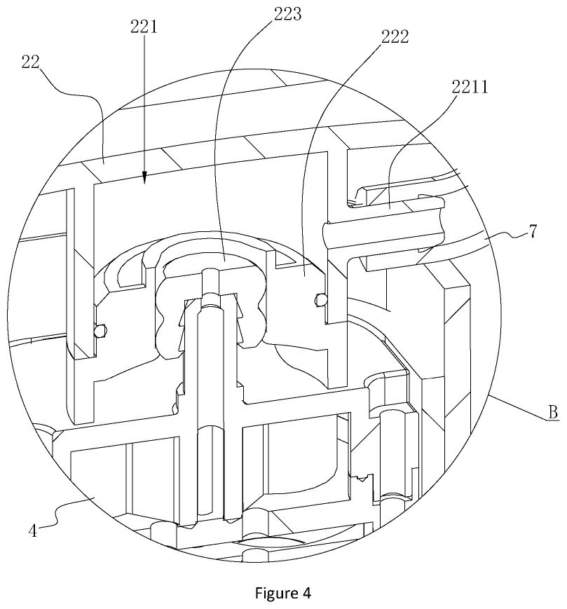

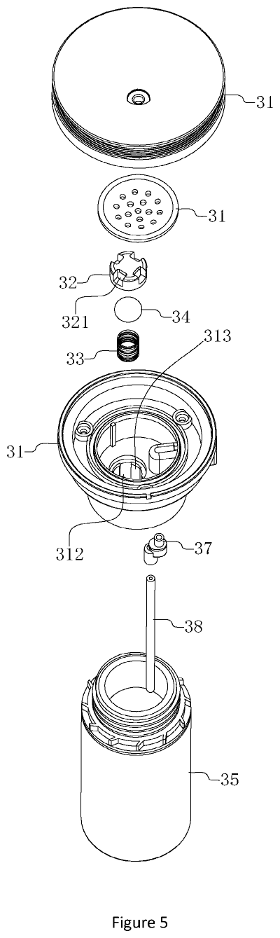

Please refer to FIGS. 1-5, the aroma diffuser of the present embodiment comprises an atomization mechanism 3, wherein the atomization mechanism 3 serves as a basic functional unit of the aroma diffuser and is used to atomize essential oils into mist.

The atomization mechanism 3 comprises an atomization shell 31 and an essential oil bottle 35, in the present embodiment, the atomization shell 31 comprises a bottom shell and an upper cover integrated together, on the upper cover are provided mist outlets, and the essential oil bottle 35 is a glass bottle suitable for holding aromatherapy essential oils, the essential oil bottle 35 is provided with a first cavity 10, the atomization shell 31 is provided with a second cavity 20, and the second cavity 20 is specifically a mist outlet cavity in the atomization shell 31.

The essential oil bottle 35 and the atomization shell 31 are connected in sequence from bottom to top, so that the first cavity 10 and the second cavity 20 communicate with each other, which are connected through threads in the present embodiment;

The atomization mechanism 3 further comprises a leakage-proof cover 32, an elastic body 33 and a ball 34.

The leakage-proof cover 32 is made of engineering plastic material, the elastic body 33 comprises a spring with two end portions, and the ball 34 is a sphere, specifically one of a steel ball, a plastic ball and a glass bead.

At the atomization shell 31 is formed a leakage-proof accommodation cavity 312, specifically, the leakage-proof accommodation cavity 312 is integrally formed at a bottom portion of the atomization shell 31, and a bottle mouth of the essential oil bottle 35 can cover the leakage-proof accommodation cavity 312; at the leakage-proof accommodation cavity 312 are provided a first communication hole 313 communicated to the first cavity 10, essentially, the first communication hole 313 is a grid-like structure provided on a side wall of the leakage-proof accommodation cavity 312, and a second communication hole 321 is provided at the leakage-proof cover 32, essentially, the second communication hole 321 is a grid-like structure provided at the leakage-proof cover 32, and the anti-leakage cover shell 32 is closed at an upper opening portion of the leakage-proof accommodation cavity 312 (the anti-leakage cover shell 32 and the leakage-proof accommodation cavity 312 are interlocked or locked with screws), so as to communicate the second communication hole 321 to the second cavity 20;
the elastic body 33 and the ball 34 are arranged inside the leakage-proof accommodation cavity and the leakage-proof cover 312 from bottom to top, and an end of the elastic body 33 abuts against an inner wall of the leakage-proof accommodation cavity 312, and another end of the elastic body 33 abuts against the ball 34; and when the atomization mechanism 3 is placed normally, pressure exerted by the ball 34 on the spring compresses the spring to make the second communication hole 321 of the leakage-proof cover 32 open; when the atomization mechanism 3 is tilted, pressure exerted by the ball 34 on the spring is not sufficient to compress the spring, and the spring stretches to make the ball 34 abut against at least a portion of the leakage-proof cover 32, so that the second communication hole 321 of the leakage-proof cover 32 is closed.

Further, an opening portion of the leakage-proof cover 32 forms a abutting portion 322 matching with the ball 34, essentially, the abutting portion 322 is a curved surface that matched with the ball 34, i.e., when the ball 34 abuts against the abutting portion 322 of the leakage-proof cover 32, there is no clearance between the surface of the ball 34 and the surface of the abutting portion 322; when the atomization mechanism 3 is tilted, pressure exerted by the ball 34 on the spring is not sufficient to compress the spring, and the spring extends to make the ball 34 abut against the abutting portion 322 of the leakage-proof cover 32, thereby closing the second communication hole 321 of the leakage-proof cover 32.

Further, on a bottom inner wall of the leakage-proof accommodation cavity 312 forms support columns 314 for supporting the elastic body 33, the support columns 314 are integrally formed with the leakage-proof accommodation cavity 312, after be assembled, the support columns 314 support the elastic body 33 from inner portions of the elastic body 33 to avoid radial dislocation of the elastic body 33.

Specifically, the atomization mechanism 3 further comprises a nozzle assembly, wherein the nozzle assembly comprises an atomization nozzle 36 and an air intake nozzle 37, wherein,
the atomization nozzle 36 and the air intake nozzle 37 are work pieces made of engineering plastics, or the atomization nozzle 36 and the air intake nozzle 37 can also be metal work pieces with corrosion resistance;
the atomization nozzle 36 comprises a longitudinally arranged oil suction channel 361 and a longitudinally arranged atomization channel 363 arranged parallel to each other, and a horizontal oil suction channel 362 comprising two ends connected to the longitudinally arranged oil suction channel 361 and the longitudinally arranged atomization channel 363 respectively, and a bottom end portion of the longitudinally arranged atomization channel 363 is provided with an atomization jet orifice 364; essentially, an outer outline of the atomization nozzle 36 is roughly an “L” shape, the longitudinally arranged oil suction channel 361, the horizontally arranged oil suction channel 362 and the longitudinally arranged atomization channel 363 are all diversion channels inside the atomization nozzle 36, and the atomization jet orifice 364 is an open hole at the bottom end portion of the longitudinally arranged atomization channel 363.

Inside the air intake nozzle 37 forms an air injection channel 374, which essentially is an axial channel that traverses the air intake nozzle 37.

The air intake nozzle 37 penetrates into the longitudinally arranged atomization channel 363 of the atomization nozzle 36, so that the air injection channel 374 of the air intake nozzle 37 and the atomization jet orifice 364 of the longitudinally arranged atomization channel 363 are aligned with each other, and between the air intake nozzle 37 and the longitudinally arranged atomization channel 363 of the atomization nozzle 36 forms a gap which serves as an atomization cavity.

Further, the air intake nozzle 37 comprises a first mounting part 371, a rib portion 372, and a second mounting portion 373 sequentially arranged along an axial direction thereof, essentially, an outer profile of the air intake nozzle 37 is cylindrical, an outer diameter of the first mounting portion 371 is approximately same as an outer diameter of the second mounting portion 373, while an outer diameter of the rib portion 372 is larger than outer diameters of the first mounting portion 371 and the second mounting portion 373; the first mounting portion 371 of the air intake nozzle 37 penetrates into the longitudinally arranged atomization channel 363 of the atomization nozzle 36 (through interference fit or riveting), and the rib portion 372 of the air intake nozzle 37 is designed to abut against a port of the longitudinally arranged atomization channel 363 of the atomization nozzle 36, and the second mounting portion 373 of the air intake nozzle 37 is suitable for inserting into a structure.

Further, an outlet end of the air injection channel 374 is narrowed into a truncated cone shape or a cone shape to increase an airflow rate through the outlet end of the air injection channel 374.

An airflow channel 311 is formed in the atomization shell 31, the airflow channel 311 is integrally formed with the atomization shell 31, an inlet of the airflow channel 311 is located outside the atomization shell 31, and an outlet of the airflow channel 311 faces the essential oil bottle 35; a free end of the air inlet nozzle 37 (i.e., the second mounting portion 373) is inserted into an outlet of the air flow channel 311 of the atomization shell 31, and the two have an interference fit, so that the nozzle assembly is supported in the first cavity 10 of the essential oil bottle 35, an oil suction pipe 38 is inserted at and connect to the longitudinally arranged oil suction channel 361 of the atomization nozzle 36, the two have an interference fit, and the free end of the oil suction pipe 38 enters the essential oil bottle 35.

Specifically, the aroma diffuser of the present embodiment further comprises an outer shell mechanism 1, an inner support frame mechanism 2 and an air pump 4, wherein
the shell mechanism 1 includes a base 12 and a shell body 11 fixedly arranged in sequence from bottom to top, and the shell body 11 has an inner and outer two-layer structure nested with each other, the air pump 4 is a compressed air pump 4 driven by a DC motor or an impeller air pump 4, which can output airflow to the outside; and
the inner support frame mechanism 2 comprises a lower support frame 21 and an upper support frame 22, the lower support frame 21 and the upper support frame 22 are both engineering plastic support frames, and the lower support frame 21 and the upper support frame 22 are fixed to each other (through screws or fasteners) and accommodated in the outer shell mechanism 1; the air pump 4 is fixed in the lower support frame 21 (specifically through screws or fasteners), and an air outlet end of the air pump 4 is connected into a silencing cavity 221, and a first joint 2211 is provided at the silencing cavity 221; the upper support frame 22 is provided with a second joint 224, the second joint 224 is an independent joint and is embedded near an opening of the upper support frame 22, and the second joint 224 is connected to the first joint 2211 of the silencing cavity 221 through an air pipe 7; and the atomization shell 31 of the atomization mechanism 3 is adapted to be plugged into the upper support frame 22 of the inner support frame mechanism 2, the openings of which are friction-fitted in the present embodiment, and the atomization shell 31 of the atomization mechanism 3 is plugged into and connected to the upper support frame 22 of the inner support frame mechanism 2, and the free end of the second joint 224 is connected to the inlet of the airflow channel 311 of the atomization shell 31.

Further, specifically, the silencing cavity 221 is formed at a bottom portion of the upper support frame 22, that is, the silencing cavity 221 is surrounded by an enclosure formed by pulling up bottom portions of the upper support frame 22, and the first joint 2211 is integrally formed on a side wall of the silencing cavity 221, an opening portion of the silencing cavity 221 is covered with a silencing cavity sealing plug 222, the silencing cavity sealing plug 222 is an engineering plastic cover body, which is matched with an opening of the silencing cavity 221, and the air outlet end of the air pump 4 is connected into the silencing cavity sealing plug 222 and thereby into the silencing cavity 221.

Further, specifically, between the air outlet end of the air pump 4 and the silencing cavity sealing plug 222 is provided an air outlet buffer body 223, essentially, the air outlet buffer body 223 is made of rubber, silicone or soft plastic, comprising a circular outer contour, the air outlet buffer body 223 penetrates into an end face of the silencing cavity sealing plug 222, and the air outlet end of the air pump 4 is connected to an axial through hole of the air outlet buffer body 223 so as to be connected to the silencing cavity sealing plug 222.

Further, between the air pump 4 and an inner wall of the lower support frame 21 is provided an air pump buffer body 211, essentially, the air pump buffer body 211 is made of silencer foam, rubber, silicone or soft plastic, comprising a circular outer outline, the air pump buffer body 211 is arranged around a side of the air pump 4, and the air pump buffer body 211 abuts against the inner wall of the lower support frame 21.

Understandably, the aroma diffuser further comprises batteries 5 and a circuit board 6, the batteries 5 are rechargeable lithium batteries 5, the circuit board 6 is a printed circuit board 6 (PCB), and the circuit board 6 is equipped with a main control. In the present embodiment, the batteries 5 are fixed in the lower support frame 21 of the inner support frame mechanism 2, and the circuit board 6 is fixed in the base 12; the batteries 5 and the air pump 4 are electrically connected to the circuit board 6, which are electrically connected through wires.

Understandably, the circuit board 6 is equipped with a charging interface (not shown in the figures), which is one of the Type-C interface, MicroUSB interface and MiniUSB interface. The charging interface is inserted from the base 12 to charge the batteries 5.

Understandably, the circuit board 6 is also equipped with a switch (not shown in the figures), and the base 12 is embedded with a button aligned with the switch (not shown in the figures). When the button is pressed, the switch can be triggered to control on-and-off, mode switching, timing and running gear of the aroma diffuser.

When the aroma diffuse of the present embodiment is placed normally, pressure exerted by the ball 34 on the spring compress the spring to make the second communication hole 321 of the leakage-proof cover 32 open; after the air pump 4 is powered and starts running, and the air outlet thereof begins to output airflow externally, when the airflow enters the silencing cavity 221, at this time, noise generated when the air pump 4 discharges air will be eliminated by the silencing cavity 221; furthermore, vibration generated when the air pump 4 runs will be eliminated by the outlet buffer body 223 and the air pump buffer body 211, in this way, noise and vibration generated when the air pump 4 discharges air can be eliminated; the airflow in the silencing cavity 221, driven by air pressure, exits the silencing cavity 221 through the first joint 2211, passes through the air pipe 7, and then through the second joint 224, enters into the airflow channel 311 of the atomization shell 31; due to the insertion of the free end of the air intake nozzle 37 (i.e., the second mounting portion 373) into the outlet of the airflow channel 311 of the atomization shell 31, the airflow channel 311 of the atomization shell 31 is communicated to the air jet channel 374 of the air intake nozzle 37, and the airflow passes successively through the air injection channel 374 of the air intake nozzle 37, the gap between the atomization nozzle 36 and the air intake nozzle 37, and the atomization jet orifice 364 of the atomization nozzle 36; at this point, due to the airflow in the gap between the atomization nozzle 36 and the air intake nozzle 37 has a high-speed, aromatic essential oils in the oil bottle 35 will slowly be drawn through the oil suction pipe 38, and the longitudinally arranged oil suction channel 361 and the horizontally arranged oil suction channel 362 of the atomization nozzle 36, mixed with the airflow in that gap, and atomized into mist; the atomized mist is sprayed towards inner sides of the oil bottle 35, and any excess aromatic essential oils will be directly liquefied in the oil bottle 35, preventing wastage; non-liquefied mist can enter the leak-proof accommodation cavity 312 through the first communication hole 313, pass through the leak-proof containment cavity 312, and then, via the second communication hole 321, reach the second cavity 20 of the atomization shell 31 (i.e., during atomization), and subsequently, be diffused into the air through the atomization orifice of the atomization shell 31. When the aroma diffuser is tilted, due to an angle existing between the gravity of the ball 34 and a rebound force of the spring, pressure exerted by the ball 34 on the spring is not sufficient to compress the spring, so as to cause the spring extend, so that the ball 34 abuts against the abutting portion 322 of the leak-proof cover 32, at this point, there is no gap between the surface of the ball 34 and the surface of the abutting portion 322, liquid is prevented from passing through, and the second communication hole 321 of the leak-proof cover 32 is closed. With the setup, even if the aroma diffuser is tilted, aromatic essential oils cannot pass through the leak-proof cover 32, thereby preventing the leakage of aromatic essential oils from the aroma diffuser.

In the aroma diffuser of present embodiment, through the coordination of the elastic body 33 and the ball 34, when tilted, the essential oils cannot pass through the leakage-proof cover 32, thereby preventing leaks from the aroma diffuser, enhancing its safety during use. Moreover, by incorporating the silencing cavity 221, the air outlet buffer body 223, and the air pump buffer body 211, noise and vibration during air pump operation can be eliminated, making the aroma diffuser quieter when running. Additionally, the gap serving as an atomization cavity between the air intake nozzle 37 and the longitudinally arranged atomization channel 363 of the atomization nozzle 36 reduces the volume of the atomization cavity, making the aroma diffuser more compact. The atomization jet orifice 364 of the atomization nozzle 36 is oriented towards inner sides of the essential oil bottle, effectively reducing noise and preventing wastage of essential oils, providing the advantage of lower aroma consumption. In conclusion, the present invention resolves a series of problems arising from enlargement, providing an adaptive structure for the diffuser's enlargement.

Although the embodiments of the present invention have been shown and described, those of ordinary skill in the art will understand that various changes and modifications can be made to these embodiments without departing from the principles and spirit of the present invention, the scope of which is defined by the appended claims and their equivalents.

1. An aroma diffuser, comprising an atomization mechanism, wherein
the atomization mechanism comprises an atomization shell and an essential oil bottle, the essential oil bottle is provided with a first cavity, and the atomization shell is provided with a second cavity;
the essential oil bottle and the atomization shell are connected in sequence from bottom to top, so that the first cavity and the second cavity communicate with each other;
the atomization mechanism further comprises a leakage-proof cover, an elastic body and a ball;
at the atomization shell is formed a leakage-proof accommodation cavity, at the leakage-proof accommodation cavity is provided a first communication hole communicated to the first cavity, at the leakage-proof cover is provided a second communication hole, and the anti-leakage cover shell is closed at an upper opening portion of the leakage-proof accommodation cavity so as to communicate the second communication hole to the second cavity;
the elastic body and the ball are arranged inside the leakage-proof accommodation cavity and the leakage-proof cover from bottom to top, an end of the elastic body abuts against an inner wall of the leakage-proof accommodation cavity, and another end of the elastic body abuts against the ball; and
when the atomization mechanism is placed normally, pressure exerted by the ball on a spring compresses the spring to make the second communication hole of the leakage-proof cover open; when the atomization mechanism is tilted, pressure exerted by the ball on the spring is not sufficient to compress the spring, and the spring stretches to make the ball abut against at least a portion of the leakage-proof cover, so that the second communication hole of the leakage-proof cover is closed.

2. The aroma diffuser according to claim 1, wherein an opening portion of the leakage-proof cover forms a abutting portion matching with the ball; and
when the atomization mechanism is tilted, pressure exerted by the ball on the spring is not sufficient to compress the spring, and the spring extends to make the ball to abut against the abutting portion of the leakage-proof cover, thereby closing the second communication hole of the leakage-proof cover.

3. The aroma diffuser according to claim 1, wherein on a bottom inner wall of the leakage-proof accommodation cavity forms a support column for supporting the elastic body.

4. The aroma diffuser according to claim 1, wherein the atomization mechanism further comprises a nozzle assembly,
the nozzle assembly comprises an atomization nozzle and an air intake nozzle;
the atomization nozzle comprises a longitudinally arranged oil suction channel and a longitudinally arranged atomization channel arranged parallel to each other, and a horizontally arranged oil suction channel comprising two ends connected to the longitudinally arranged oil suction channel and the longitudinally arranged atomization channel respectively, and an bottom end portion of the longitudinally arranged atomization channel is provided with an atomization jet orifice;
inside the air intake nozzle forms an air injection channel;
the air intake nozzle penetrates into the longitudinally arranged atomization channel of the atomization nozzle, so that the air injection channel of the air intake nozzle and the atomization jet orifice of the longitudinally arranged atomization channel are aligned with each other, and between the air intake nozzle and the longitudinally arranged atomization channel of the atomization nozzle is a gap which serves as an atomization cavity;
an airflow channel is formed in the atomization shell, an inlet of the airflow channel is located outside the atomization shell, and an outlet of the airflow channel faces the essential oil bottle; and
a free end of the air inlet nozzle is inserted into an outlet of the air flow channel of the atomization shell, so that the nozzle assembly is supported in the first cavity of the essential oil bottle, an oil suction pipe is inserted at and connected to the longitudinally arranged oil suction channel of the atomization nozzle, and the free end of the oil suction pipe enters the essential oil bottle.

5. The aroma diffuser according to claim 4, wherein an outlet end of the air injection channel is narrowed into a truncated cone shape or a cone shape.

6. The aroma diffuser according to claim 1, further comprising an outer shell mechanism, an inner support frame mechanism and an air pump, wherein
the inner support frame mechanism comprises a lower support frame and an upper support frame, and the lower support frame and the upper support frame are fixed to each other and accommodated in the outer shell mechanism;
the air pump is fixed in the lower support frame, and an air outlet end of the air pump is connected into a silencing cavity, and a first joint is provided at the silencing cavity;
the upper support frame is provided with a second joint, and the second joint is connected to the first joint of the silencing cavity through an air pipe; and
the atomization shell of the atomization mechanism is adapted to be plugged into the upper support frame of the inner support frame mechanism, and the atomization shell of the atomization mechanism is plugged into and connected to the upper support frame of the inner support frame mechanism, so that, the free end of the second joint is connected to the inlet of the airflow channel of the atomization shell.

7. The aroma diffuser according to claim 6, wherein the silencing cavity is formed at a bottom portion of the upper support frame, an opening portion of the silencing cavity is covered with a silencing cavity sealing plug, and the air outlet end of the air pump is connected into the silencing cavity sealing plug and thereby into the silencing cavity.

8. The aroma diffuser according to claim 7, wherein between the air outlet end of the air pump and the silencing cavity sealing plug is provided an air outlet buffer body.

9. The aroma diffuser according to claim 6, wherein between the air pump and an inner wall of the lower support frame is provided an air pump buffer body.

10. The aroma diffuser according to claim 6 further comprising batteries and a circuit board, wherein both the batteries and the circuit board are accommodated in the outer shell mechanism, and
the batteries and the air pump are both electrically connected to the circuit board.
